(19) United States
(12) Patent Application Publication
(10) Pub. No.: US 2009/0319046 A1
(43) Pub. Date: Dec. 24, 2009

(54) HYOID SUSPENSION FOR OBSTRUCTIVE SLEEP APNEA

(75) Inventors: Yosef P. KRESPI, Ridgefield, CT (US); Craig D. FRIEDMAN, Westport, CT (US); Arindam DATTA, Hillsborough, NJ (US); Lawrence P. LAVELLE, Rahway, NJ (US)

Correspondence Address: William H. Dippert;Eckert Seamans Cherin & Mellott, LLC, U.S. Steel Tower, 600 Grant Street, 44th Floor, Pittsburgh, PA, 15219, US

(73) Assignee: BIOMERIX CORPORATION, New York, NY (US)

(21) Appl. No.: 12/490,280

(22) Filed: Jun. 23, 2009

Related U.S. Application Data
(60) Provisional application No. 61/074,728, filed on Jun. 23, 2008.

(51) Int. Cl. A61F 2/28; A61B 17/56

(52) U.S. Cl. 623/16.11; 128/898

(57) ABSTRACT

In an improved method for surgically treating a sleep-related breathing disorder, comprising hyoid distruction/expansion and hyo-mandibular suspension, an implant comprises a polypropylene and/or reticulated elastomeric biostable polyurethane matrix. The matrix is positioned between two or more segments of the distructed hyoid bone. A single or multiple midline or paramedian incisions are made under a patient's chin; soft tissues overlying the mandible are cleaned; a first screw is inserted into the edge of the mandible; a second screw is inserted into the inferior edge of the mandible; a second horizontal incision is made over the body of the hyoid; the infrahyoid muscles are separated from the body of the hyoid bone; the hyoid is refracted and stabilized; the sternohyoid and thyrohyoid muscles are detached from the body of the hyoid between the lesser cornuae; the hyoid bone is divided midline or paramedian into two or more segments; and an absorbable or nonabsorbable, rigid or semirigid, implant is positioned between segments of the hyoid bone. The hyoid bone is stabilized by the implant; and sutures or portions of polypropylene and/or polyurethane matrix are passed around the hyoid bone segments and implant to connect to muscle still attached to the upper portion of the hyoid bone, wherein the hypopharyngeal airway is expanded.

### FIELD OF THE INVENTION

This invention is directed to a method of treating sleep disorders. More particularly, this invention is directed to a method of treating sleep apena comprising hyoid expansion and hyo-mandibular suspension.

### BACKGROUND OF THE INVENTION

Upper airway obstruction results from excess and/or collapse of soft tissue in the soft palate, tonsillar pillars, tongue, tongue base, and hypopharyngeal walls. Specific anatomic risk factors predisposing to obstructive sleep apnea/hypopnea syndrome (OSAHS) include long soft palate, shallow palatal arches, large tongue base, narrow mandibular arches, and mandibular hypoplasia. [Rojewski et al., 1984] Other general anatomic factors often associated with OSAHS include obesity, large body mass index (BMI), shortened thick neck, anterior larynx location, enlarged tonsils and/or adenoids, lingual tonsils, thickened pharyngeal walls, elongated uvula, redundant soft palate, large tongue volume, deviated nasal septum, turbinate hypertrophy, and redundant or folded epiglottis. [Chervin et al., 1996] Cephalometric studies employing computed tomography (CT) have correlated increased OSAHS severity with increased BMI, larger tongue and soft palate volumes, and decreased airway space. [Lowe et al., 1995] Diagnostically, Fujita employed endoscopy with the Mueller maneuver (reverse Valsalva) to identify sites of obstruction in the nasopharynx, oropharynx and hypopharynx. [Fujita et al., 1987] Three general profile types were identified based on the predominant areas of obstruction:

Type I—retropalatal/velopharyngeal;

Type II—retropalatal/velopharyngeal and hypopharyngeal; and

Type III—hypopharyngeal.

Catheter pressure transducers employed in OSA sleep studies have shown that upper airway collapse occurred at the velopharyngeal/retropalatal level and the hypopharyngeal/postlingual level in comparable frequencies. [Chaban et al., 1988]

The most commonly used grading system to identify retrolingual obstruction is the Friedman tongue position (FTP) (see, M. Friedman, Multilevel Pharyngeal Surgery, Elsevier Health Sciences, 2008, Chapter 1). Patients with FTP III and IV have a likelihood of retrolingual obstruction contributing to OSAHS.

### SUMMARY OF THE INVENTION

According to the invention, a method for surgically treating a sleep-related breathing disorder comprises hyoid expansion and hyo-mandibular suspension. In one embodiment, the method also includes transoral or transcervical tongue suspension. In another embodiment of the invention, a method for treating sleep disorders comprises making an incision under a patient's chin; cleaning soft tissues overlying the mandible; inserting a first screw into the mandible; inserting a second screw into the inferior edge of the mandible; making a second horizontal incision over the body of the hyoid; separating the infrahyoid muscles from the body of the hyoid bone; refracting and stabilizing the hyoid; detaching the sternohyoid and thyrohyoid muscles; and suturing the suprahyoid muscles. In a further embodiment of the invention the hypopharyngeal airway is enlarged. When the hypopharyngeal airway is enlarged, the hyoid bone is divided and a cylindrical or substantially cylindrical polymeric implant is positioned between the hyoid bone segments. The implant comprises a rigid or semirigid polypropylene and/or polyurethane matrix, preferably a reticulated, biodurable, elastomeric matrix made from polycarbonate polyurethane or polycarbonate polyurea-urethane.

In one aspect of the invention, an improved method for surgically treating a sleep-related breathing disorder, comprising hyoid destruction/expansion and hyo-mandibular suspension, the improvement comprises an implant comprising a polypropylene and/or reticulated elastomeric biostable polyurethane matrix with the matrix being positioned between two or more segments of the destructed hyoid bone.

In another aspect of the invention, the method also includes transoral or transcervical tongue suspension using similar implants.

In another aspect of the method of the invention wherein a single or multiple midline or paramedian incisions are made under a patient's chin;
soft tissues overlying the mandible are cleaned;
a first screw is inserted into the edge of the mandible;
a second screw is inserted into the inferior edge of the mandible;
a second horizontal incision is made over the body of the hyoid;
the infrahyoid muscles are separated from the body of the hyoid bone;
the hyoid is refracted and stabilized;
the sternohyoid and thyrohyoid muscles are detached from the body of the hyoid between the lesser cornuae;
the hyoid bone is divided midline or paramedian into two or more segments;
an absorbable or nonabsorbable, rigid or semirigid, implant is positioned between segments of the hyoid bone, whereby the hyoid bone is stabilized by the implant; and
sutures or portions of polypropylene and/or polyurethane matrix are passed around the hyoid bone segments and implant to connect to muscle still attached to the upper portion of the hyoid bone,
wherein the hypopharyngeal airway is expanded.

In another aspect of the method of the invention, the implant comprises a polypropylene and/or polyurethane matrix.

In another aspect of the method of the invention, the implant is cylindrical or substantially cylindrical with indentations at its respective ends.

In another aspect of the method of the invention, the implant substantially conforms to the substantially conform to the ends of the hyoid segments.

In another aspect of the method of the invention, the implant is attached to one or more hyoid bone segments with one or more staples, another mechanical means, or adhesive.

### BRIEF DESCRIPTION OF THE DRAWINGS

FIG. 1 is a view of the insertion of two Repose screws to the mental (?) area of the mandible via stab incision. An insert shows details of a screw with preloaded suture;

FIG. 2 is a view of a horizontal incision exposing the body of the hyoid, where infrahyoid muscle is separated with electrocautery;

FIG. 3 depicts delivery of sutures to the lower incision with a suture passer;

FIG. 4 is a view of hyoid distraction, division of the hyoid body in the midline after myotomy using a bone cutter or chisel;

FIG. 5 represents division of the hyoid bone in the midline and suspension of the hyoid segments toward the mandible by figure eight sutures;

FIG. 6 is a view of hyoid spacer between the two cut ends of the bone, using nonabsorbable graft, resulting in hyoid expansion;

FIG. 7 is a schematic representation of a hyoid spacer useful according to the invention; and FIG. 7A is a cross-sectional view of the spacer in FIG. 7 across lines 7A-7A;

FIGS. 8A and 8B are lateral and top views of another embodiment of a tongue retractor;

FIG. 9 is a schematic representation of one embodiment of a tongue retractor;

FIGS. 10A and 10B are lateral and top views of yet another embodiment of the invention; and

FIGS. 11 and 12 are each a micrograph representing tissue and cell ingrowth on a hyoid spacer implanted according to the invention.

### DETAILED DESCRIPTION OF THE INVENTION

In the event of severe OSAHS, hyoid expansion and hyomandibular suspension procedure is performed. These surgical methods enhance the anterior superior repositioning of the tongue base, enlarge the pharyngeal airway in a lateral dimension, and partially separate the tongue base from the lower airway by an infrahyoid myotomy. Hyoid expansion and hyo-mandibular suspension/myotomy may be performed in conjunction with or following transoral or transcervical tongue suspension.

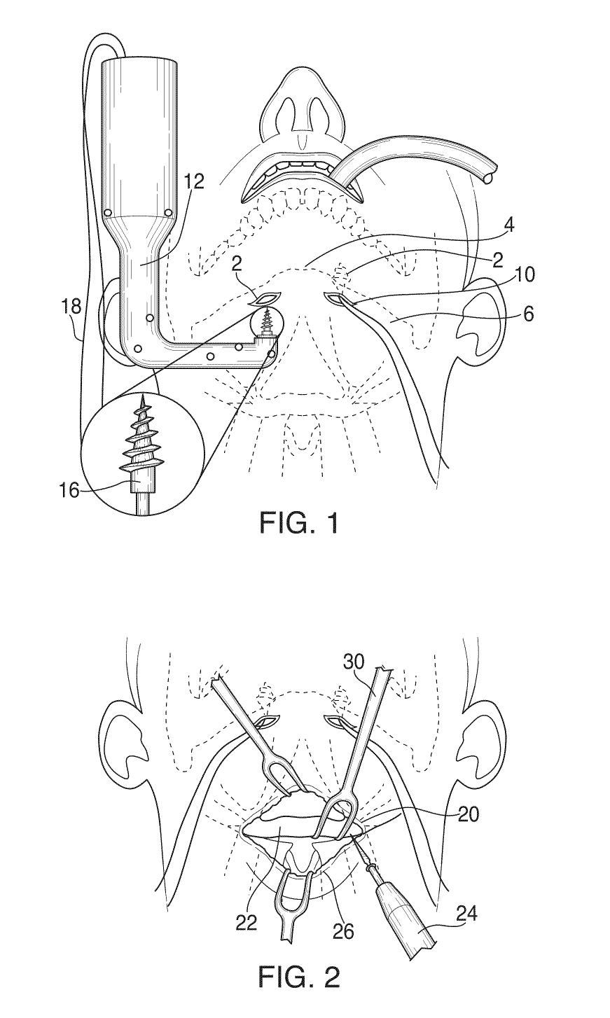

In a procedure according to the invention, a patient's anterior neck is prepped and draped. The anterior mandible, hyoid, and the thyroid cartilages are then outlined with a skin marker with the neck slightly extended. Lidocaine with epinephrine 1:100,000 is injected into the planned incision sites. As shown in FIG. 1, two about 0.5 cm incisions 2 are made under a patient's chin 4 off the midline, and, by use of blunt dissection, the soft tissues overlying the mandible 6 are cleaned. A first screw 10 is inserted using a screw inserter 12, such as, for example, a REPOSE™ screw inserter (Influent, Concord, N.H.), after which the screw inserter 12 is loaded with a second screw 16. Inserter 12 is positioned perpendicular to mandible 6, firm pressure is applied, and screw 16 is inserted into the inferior edge of mandible 6. A loop of #1 polypropylene suture 18 is preloaded to screw 16 by the manufacturer. In another embodiment, loop 18 can be made from other biostable materials, such as polyolefins, polyamides, polyesters, polyurethanes, and the like.

A second horizontal incision 20 measuring about 5 to 6 cm is made over the body of the hyoid 22, as shown in FIG. 2. Subcutaneous fatty adipose tissue can be dissected and removed. An electrocautery device 24 is used to separate the infrahyoid muscles 26 from the body of the hyoid bone 22. A single bone hook 30 is placed to retract and stabilize the hyoid 22 during the dissection. The sternohyoid and thyrohyoid muscles are detached from the body of the hyoid 22 between the lesser cornuae 28.

Careful dissection around the body of the hyoid eliminates risk of injury to the neurovascular structures. Careful dissection and good hemostasis is mandatory to avoid injury to the pre-epiglottic fat pad.

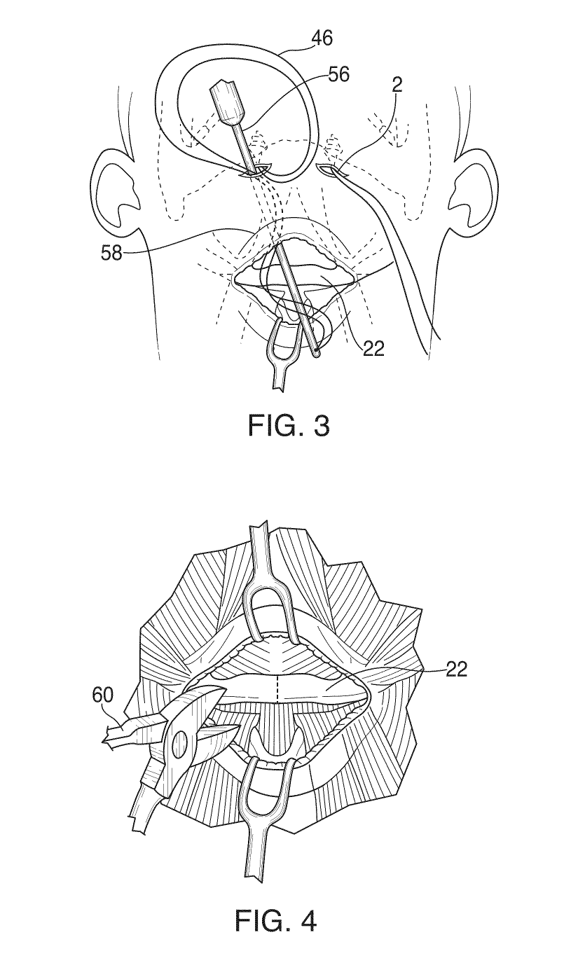

A suture passer is then loaded with polypropylene suture 46 and tunneled at the subplatysma layer from the mandibular incisions 2 into the lower (hyoid) incision 20, as shown in FIG. 3. The suture passer is then removed. One free end of polypropylene suture 46 is then loaded into a Mayo needle 56 and is passed through the suprahyoid muscles 58, catching a full thickness bite of the tissue.

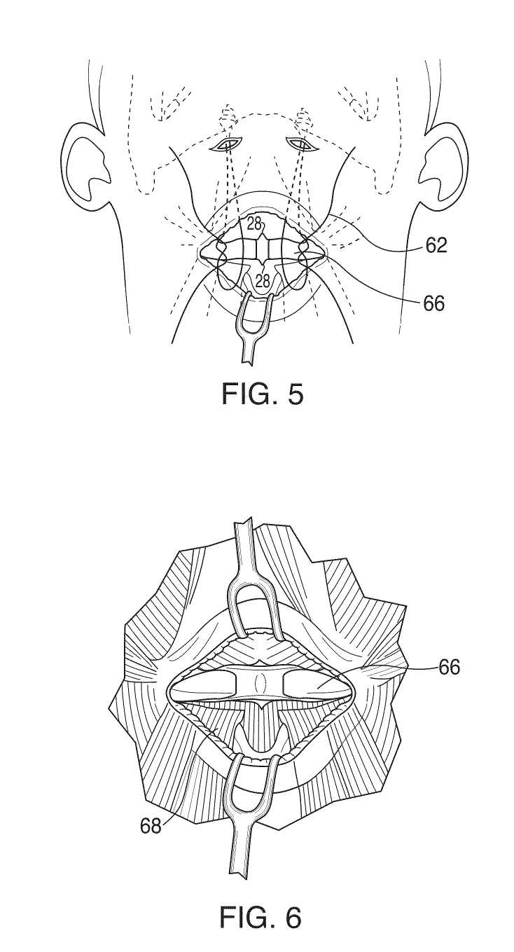

When the hypopharyngeal airway needs to be enlarged in a lateral direction, as determined by preoperative fiber optic endoscopy, the hyoid bone is then divided in the midline (hyoid distraction). Following the division of the hyoid bone 22 with an appropriate cutting instrument 60, as shown in FIG. 4, suture 62, optionally polypropylene suture, is passed around both sides of the divided hyoid segments 66 in a figure eight configuration, as shown in FIG. 5. The divided hyoid segments 66 are then prepared for suspension toward the mandible. To maintain stability of the two hyoid segments 66, an absorbable or nonabsorbable, rigid or semi rigid implant, such as an implant or hyoid spacer 68 comprising a polypropylene and/or reticulated elastomeric biodurable matrix, is placed to keep the two segments 66 of hyoid 22 separated (hyoid expansion). See FIG. 6.

Preferably hyoid spacer 68 is cylindrical or substantially cylindrical in shape and has indentations that receive and hold ends of the hyoid segments. In another embodiment, the matrix can be cylindrical, substantially cylindrical or other shapes such as elliptical or helical but can substantially conform to the ends of the hyoid segments. Optionally the matrix can be attached or affixed to the hyoid bone segments by one or more staples, sutures, clips, other mechanical means, and/or adhesive.

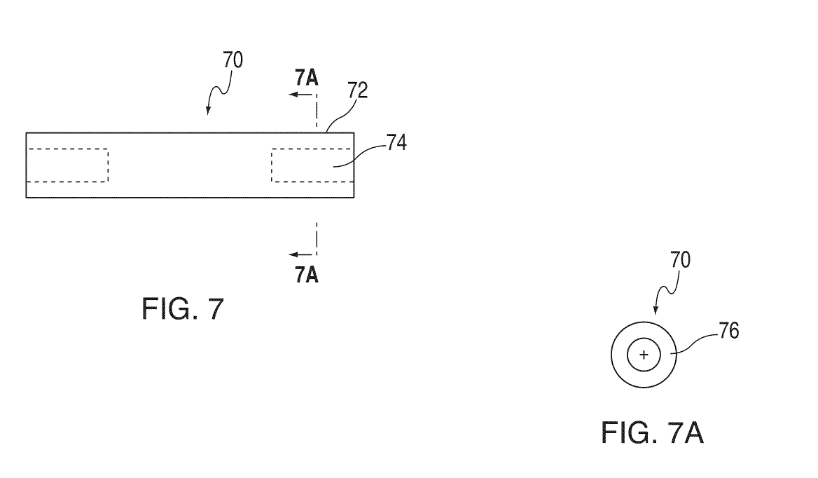

FIG. 7 is a schematic, cross-sectional representation of a hyoid spacer useful according to the invention. Typically, a hyoid spacer 70 will be a cylindrical, substantially cylindrical, or at least partially curvilinear member of from about 30 to about 50 mm, preferably from about 35 to about 45 mm, in length. Each end 72 of spacer 70 has an annular recess 74 of from about 6 to about 14 mm, preferably from about 8 to about 12 mm, in depth. As shown in FIG. 7A, annular recess 74 can have a wall thickness 76 of from about 1.5 to about 8 mm, preferably from about 3.5 to about 4.5 mm. In another embodiment, annular recess 74 can have a wall thickness of from about 3 to about 5 mm.

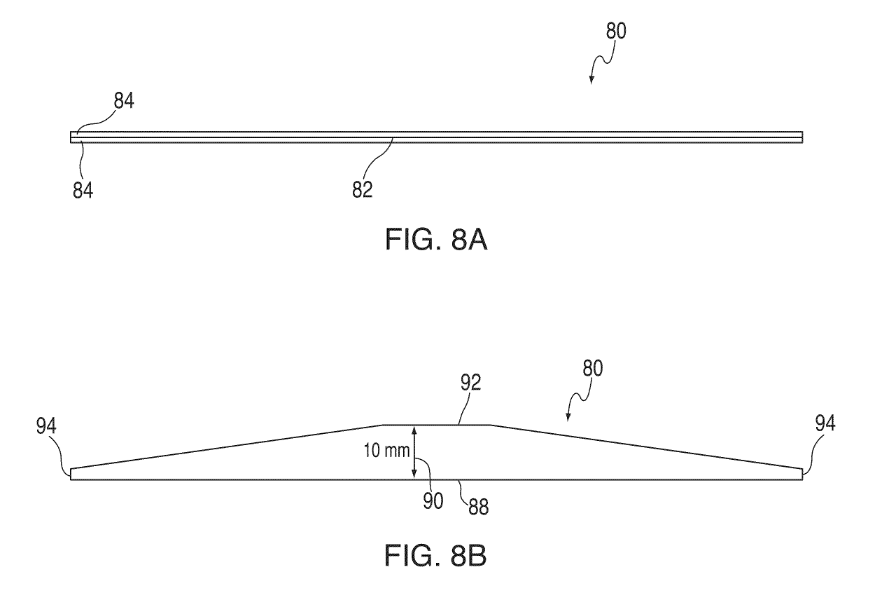

Different embodiments of tongue retractors are useful according to the invention. In an embodiment shown in FIGS. 8A and 8B, a tongue retractor 80 comprises a flat sheet 82 of biomedical grade polypropylene mesh between two flat sheets 84 of reticulated polymeric matrix. FIG. 8A is a lateral, somewhat cross-sectional view of tongue retractor 80. FIG. 8B is a top view of tongue retractor 80 where the shape and dimensions can be appreciated. Longitudinally extending side 88 has a length of from about 110 to about 130 mm, preferably from about 115 to about 125 mm, and width 90 is from about 5 to about 15 mm, preferably from about 8 to about 12 mm. The shorter lateral surface 92 is from about 10 to about 30 mm, preferably from about 15 to about 25 mm, in length, and each end surface 94 is from about 1 to about 3 mm, preferably from about 1.5 to about 2.5 mm, in length. Preferably the thickness of tongue retractor 80 is from about 1 to about 3 mm, preferably from about 1.5 to about 2.5 mm.

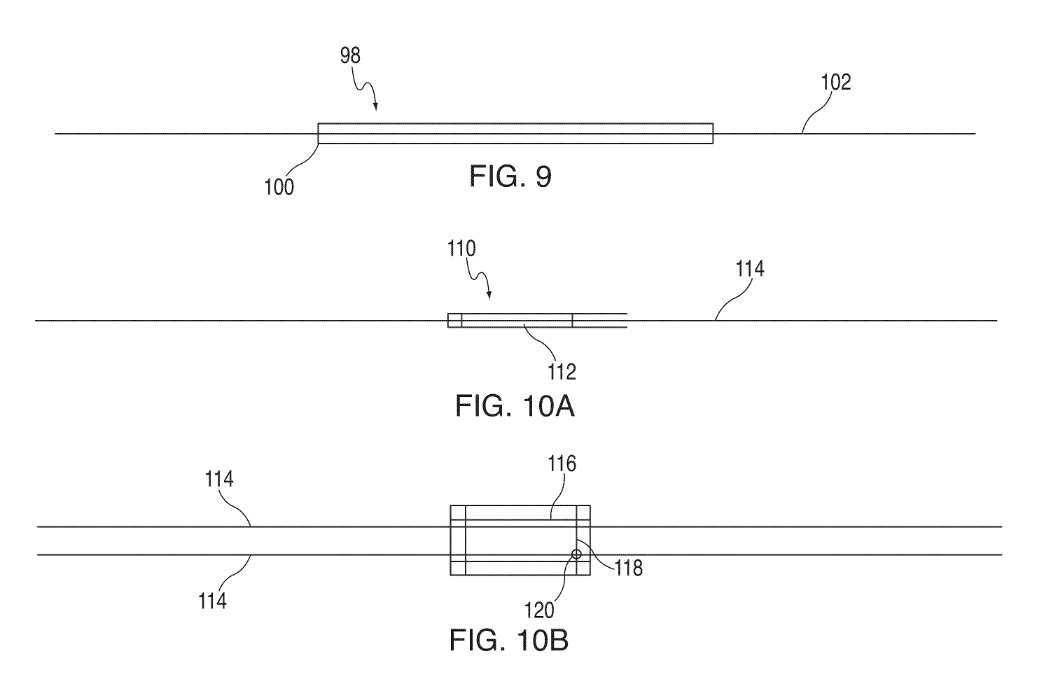

In another embodiment of a tongue retractor useful according to the invention shown in FIG. 9, a matrix protected suture configuration 98 comprises a reticulated polymeric matrix cylinder 100 with a suture 102 extending longitudinally there through. In another embodiment, reticulated polymeric matrix cylinder 100 is elastomeric, biodurable, and resilient. Cylinder 100 has a length of from about 40 to about 80 mm, preferably from about 50 to about 70 mm, and its diameter is from about 2 to about 4 mm, preferably from about 2.5 to about 3.5 mm. The suture 102, such as, for example, 2-0 Ethibond suture, has a length of from about 100 to about 140 mm, preferably from about 110 to about 130 mm.

In a further embodiment of a tongue retractor useful according to the invention shown in FIGS. 10A and 10B, tongue retractor 110 comprises a single sheet 112 of reticulated polymeric matrix having sutures 114 extending there through. In another embodiment, reticulated polymeric matrix single sheet 112 is elastomeric, biodurable, and resilient. As shown in lateral view of FIG. 10A and the top view of FIG. 10B, sutures 114 extend through sheet 112 in parallel fashion in a longitudinal direction. Suture lines 116 and 118 represent lines of suture sewn into sheet 112 in longitudinal and perpendical directions, respectively. Suture lines 118 and suture 114 knot together at intersection points 120. Each suture 114 can be, for example, 2-0 Ethibond suture, whereas each of suture lines 116 and 118 can be, for example, 5-0 Ethibond suture.

Each end of each suture 114 extends from about 40 to about 80 mm, preferably from about 50 to about 70 mm, from sheet 112. Sheet 112 has a thickness of from about 1 to about 3 mm, preferably from about 1.5 to about 2.5 mm. The length of sheet 112 is from about 10 to about 30 mm, preferably from about 15 to about 25 mm, and the width of sheet 112 is from about 5 to about 15 mm, preferably from about 8 to about 12 mm. Each suture line 116 is from about 1 to about 3 mm, preferably from about 1.5 to about 2.5 mm, from the shorter edge 124 of sheet 112, and each suture line 118 is from about 2 to about 4 mm, preferably from about 2.5 to about 3.5 mm, from the longitudinal edge 126 of sheet 112.

In another embodiment of the invention, the hyoid spacer can be made from any suitable biocompatible polymer. Useful polymers include, but are not limited to, polyamides (Nylon 666, Nylon 6,6, Nylon 12, etc.), polyolefins (e.g., polypropylene, polyethylene), nonabsorbable polyesters (e.g., polyethylene terephthalate), polyurethane, polycarbonate polyurethane, polysiloxane polyurethane, polycarbonates; polyoxymethylenes; polyimides; polyethers, polyvinylidene halides such as polyvinylidene fluoride, liquid crystalline polymers, etc. In another embodiment, the hyoid spacer can be made from bioabsorbable aliphatic polyesters that include, but are not limited to, homopolymers, and copolymers of lactic acid, glycolic acid, lactide, glycolide, para-dioxanone, trimethylene carbonate, ε-caprolactone or a mixture thereof. Further, useful biocompatible polymers include film-forming bioabsorbable polymers; these include aliphatic polyesters, poly(amino acids), copoly(ether-esters), polyalkylenes oxalates, polyamides, poly(iminocarbonates), polyorthoesters, polyoxaesters including polyoxaesters containing amido groups, polyamidoesters, polyanhydrides, polyphosphazenes, biomolecules or a mixture thereof.

In yet another embodiment of the invention, the useful polymer comprises a reticulated elastomeric matrix selected from the group consisting of polycarbonate polyurethane urea, polycarbonate polyurea urethane, polycarbonate polyurethane, polycarbonate polysiloxane polyurethanes, polycarbonatepolysiloxane polyurethane ureas, polysiloxane polyurethanes, polysiloxane polyurethaneureas, polycarbonate hydrocarbon polyurethanes, polycarbonate hydrocarbon polyurethane ureas, and mixtures of two or more thereof. In yet another embodiment of the invention, the useful polymer comprises a reticulated biodurable elastomeric matrix. See, for example, co-pending, commonly assigned U.S. patent applications Ser. No. 10/749,742, filed Dec. 30, 2003, titled “Reticulated Elastomeric Matrices, Their Manufacture and Use in Implantable Devices”, Ser. No. 10/848,624, filed May 17, 2004, titled “Reticulated Elastomeric Matrices, Their Manufacture and Use In Implantable Devices”, and Ser. No. 10/990,982, filed Jul. 27, 2004, titled “Endovascular Treatment Devices and Methods”, each of which is incorporated herein by reference in its entirety.

In yet another embodiment of the invention, the useful polymer comprises a reticulated elastomeric matrix that is at least partially degradable and can be selected from the group consisting of polycaprolactone polyurethanes, polycaprolactone urea-urethanes, polycaprolactone-polyglycolide polyurethanes, polycaprolactone-polyglycolide urea-urethanes, polycaprolactone-polylactide polyurethanes, polycaprolactone-polylactide urea-urethanes, polycaprolactone-d-1 lactide polyurethanes, polycaprolactone-d-1 lactide urea-urethanes or suitable copolymers and mixtures thereof.

The inventive implantable device is made from reticulated matrix, i.e., it comprises an interconnected network of cells and pores and channels and voids that provides fluid permeability throughout the implantable device and permits cellular and tissue ingrowth and proliferation into the interior of the implantable device. The biodurable elastomeric matrix or material is considered to be reticulated because its microstructure or the interior structure comprises inter-connected and inter-communicating pores and/or voids bounded by configuration of the struts and intersections that constitute the solid structure. The continuous interconnected void phase is the principle feature of a reticulated structure. In one embodiment, the cells and pores and channels and voids are substantially accessible to permits cellular and tissue ingrowth and proliferation. In one embodiment, the reticulated structure allows for ingrowth for such tissues as fibrous tissue and/or natural fibrous tissues. In another embodiment, the reticulated structure allows for ingrowth for such tissues as fibrovascular tissues, fibroblasts, fibrocartilage cells, endothelial tissues, etc. In another embodiment, the tissue ingrowth and proliferation into the interior of the implantable device allows for bio-integration of the device to the site where the device is placed. In yet another embodiment, the tissue ingrowth and proliferation into the interior of the implantable device prevents migration and recanalization.

Scaffold materials for the implants have a reticulated structure with sufficient and required liquid permeability and thus selected to permit blood, or other appropriate bodily fluid, and cells and tissues to access interior surfaces of the implants. This happens due to the presence of inter-connected and inter-communicating, reticulated open pores and/or voids and/or channels and/or concavities that form fluid passageways or fluid permeability providing fluid access all through. These inter-connected and inter-communicating, reticulated open pores and/or cells and/or voids and/or channels and/or concavities are accessible for fluid passageways or fluid permeability providing fluid access all through. The accessible and inter-connected and inter-communicating nature of the reticulated matrix distinguishes it from porous materials and in porous materials although there are voids, not all of them are accessible as they are not all inter-communicating and inter-connected as is the case with reticulated matrix. Over time, the tissue ingrowth and proliferation into the interior of the implantable device placed at the defect site leads to bio-integration of the device to the site where the device is placed. Without being bound by any particular theory, it is believed that the high void content and degree of reticulation of the reticulated elastomeric matrix not only allows for tissue ingrowth and proliferation of cells within the matrix but also allows for orientation and remodeling of the healed tissue after the initial tissues have grown into the implantable device. The biodurable reticulated elastomeric material that comprises the implant device will allow for tissue ingrowth and proliferation and bio-integrate the implant device to the aneurysm site. The biodurable reticulated elastomeric material that comprises the implant device allows for tissue ingrowth and will seal the aneurysm and in one embodiment provide a permanent sealing of the defect. The reticulated elastomeric matrix and/or the implantable device, over time, provides functionality or substantial functionality such as load bearing capability or the morphology and structure of the original tissue that is being repaired or replaced. Without being bound by any particular theory, it is believed that owing to the high void content of the reticulated elastomeric matrix or implantable device comprising it, once the tissue is healed and bio-integration takes place, most of the regenerated or repaired site consists of new tissue and a small volume fraction of the reticulated elastomeric matrix, or the implantable device formed from it.

The reticulated elastomeric matrix is resilient or is resilient in recovery and resilient compressibility enabling the desired collapsing of the implant for delivery and reconstitution in situ at the repair site. In one embodiment, the resilient and/or elastomeric nature of the reticulated matrix allows for substantial conformation of the device to the ends of the hyoid segments. In another embodiment, the resilient and/or elastomeric nature of the reticulated matrix allows for substantial conformation of the device to the contours of the hyoid segments or of the contours of the dissected hyoid segment.

In one other embodiment, the reticulated elastomeric matrix is biodurable or biostable. In one embodiment, the invention provides as an implantable device a biodurable, reticulated, resiliently compressible elastomeric matrix. In another embodiment, the implantable device is biodurable for at least 29 days. In another embodiment, the implantable device is biodurable for at least 2 months. In another embodiment, the implantable device is biodurable for at least 6 months. In another embodiment, the implantable device is biodurable for at least 12 months. In another embodiment, the implantable device is biodurable for longer than 12 months. In another embodiment, the implantable device is biodurable for at least 24 months. In another embodiment, the implantable device is biodurable for at least 5 years. In another embodiment, the implantable device is biodurable for longer than 5 years.

In one embodiment, the inventive implantable device reticulated elastomeric matrix is reinforced with a reinforcement. In other embodiments, the reinforcement is in at least one dimension, e.g., a 1-dimensional reinforcement (such as a fiber), a 2-dimensional reinforcement (such as a 2-dimensional mesh made up of intersecting 1-dimensional reinforcement elements), or a 3-dimensional reinforcement (such as a 3-dimensional grid). The reinforced elastomeric matrix can be made more functional for specific uses in various implantable devices by including or incorporating a reinforcement, e.g., fibers, into the elastomeric reticulated matrix. The enhanced functionalities that can be imparted by using a reinforcement include but are not limited to enhancing the ability of the device to withstand pull out loads associated with suturing during surgical procedures, the device's ability to be positioned at the repair site by suture anchors during a surgical procedure, and holding the device at the repair site after the surgery when the tissue healing takes place. In another embodiment, the enhanced functionalities can provide additional load bearing capacities to the device during surgery to facilitate the repair or regeneration of tissues. In another embodiment, the enhanced functionalities can provide additional load bearing capacities to the device, at least through the initial days following surgery, to facilitate the repair or regeneration of tissues. In another embodiment, the enhanced functionalities can provide additional load bearing capacities to the device following surgery to facilitate the repair or regeneration of tissues. In one embodiment, the reinforcements are placed between two layers of two layers of elastomeric reticulated matrix. In another embodiment, the reinforcements are sandwiched between two layers of two layers of elastomeric reticulated matrix. In another embodiment, one layer of elastomeric reticulated matrix can be reinforced with reinforcements. In another embodiment, multiple layers of elastomeric reticulated matrix can be reinforced with reinforcements.

One way of obtaining enhanced functionalities is by incorporating reinforcement, e.g., fibers, fiber meshes, wires and/or sutures, into the elastomeric matrix. Another exemplary way of obtaining enhanced functionalities is by reinforcing the matrix with at least one reinforcement. The incorporation of the reinforcement into the matrix can be achieved by various ways, including, but not limited to, stitching, sewing, weaving and knitting. In one embodiment, the attachment of the reinforcement to the matrix can be through a sewing stitch. In another embodiment, the attachment of the reinforcement to the matrix can be through a sewing stitch that includes an interlocking feature. In another embodiment, the attachment of the reinforcement to the matrix can be through an adhesive, a polymeric adhesive, a thermoplastic polymer that can be bonded to both the reinforcement and the matrix by compression molding process or compressive molding process. In another embodiment, the incorporation of the reinforcement into the matrix can be achieved by foaming of the elastomeric matrix ingredients around a pre-fabricated or pre-formed reinforcement element made from a reinforcement and reticulating the composite structure thus-formed to create an intercommunicating and interconnected pore structure. In one embodiment, the reinforcement used does not interfere with the matrix's capacity to accommodate tissue ingrowth and proliferation.

The reinforcement can comprise mono-filament fiber, multi-filament yarn, braided multi-filament yarns, commingled mono-filament fibers, commingled multi-filament yarns, bundled mono-filament fibers, bundled multi-filament yarns, and the like. The reinforcement can comprise an amorphous polymer, semi-crystalline polymer, e.g., polyester or nylon, carbon, e.g., carbon fiber, glass, e.g., glass fiber, ceramic, cross-linked polymer fiber, and the like, or any mixture thereof. The fibers can be made from absorbable or non-absorbable materials. In one embodiment, the fiber reinforcement of the present invention is made from a biocompatible material(s).

In one embodiment, the reinforcement can be made from at least one non-absorbable material, such as a non-biodegradable or non-absorbable polymer. Examples of suitable non-absorbable polymers include, but are not limited to, polyesters (such as polyethylene terephthalate and polybutylene terephthalate); polyolefins (such as polyethylene and polypropylene including atactic, isotactic, syndiotactic, and blends thereof as well as, polyisobutylene and ethylene-alpha-olefin copolymers); polyvinylidene halides (such as polyvinylidene fluoride and polyvinylidene chloride); polyamides (such as nylon 4, nylon 6, nylon 66, nylon 610, nylon 11, nylon 12 and polycaprolactam); alkyd resins; polycarbonates; polyoxymethylenes; polyimides; polyethers; epoxy resins; polyurethanes; rayon; rayon-triacetate; and any mixture thereof.

In another embodiment, the reinforcement can be made from at least one biodegradable, bioabsorbable or absorbable polymer. Examples of suitable absorbable polymers include but are not limited to aliphatic polyesters, e.g., homopolymers and copolymers of lactic acid, glycolic acid, lactide, glycolide, para-dioxanone, trimethylene carbonate, ε-caprolactone, polyoxaesters including polyoxaesters containing amido groups, polyanhydrides,and blends thereof. Aliphatic polyesters, for the purpose of this application, include polymers and copolymers of lactide (which includes lactic acid d-, l- and meso lactide), ε-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, 6,6-dimethyl-1,4-dioxan-2-one, and any mixture thereof.

Such fiber(s)/yarn(s) can be made by melt extrusion, melt extrusion followed by annealing and stretching, solution spinning, electrostatic spinning, and other suitable methods. Each fiber can be bi-layered, with an inner core and an outer sheath, or multi-layered, with inner core, an outer sheath and one or more intermediate layers.

The reinforcement can be made from carbon, glass, a ceramic, bioabsorbable glass, silicate-containing calcium-phosphate glass, or any mixture thereof. The calcium-phosphate glass, the degradation and/or absorption time in the human body of which can be controlled, can contain metals, such as iron, magnesium, sodium, potassium, or any mixture thereof.

The reinforcement can be incorporated into the reticulated elastomeric matrix in different patterns. In one embodiment, the reinforcement is present as a plurality of parallel and/or substantially parallel 1 dimensional reinforcement elements, e.g., as a plurality of parallel lines such as parallel fibers. In another embodiment, the reinforcement is placed as a 2- or 3-dimensional reinforcement grid in which the 1-dimensional reinforcement elements cross each other's path. The grid can have one or multiple reinforcement elements. In 2- or 3-dimensional reinforcement grid embodiments, the elements of the reinforcement can be arranged in geometrically-shaped patterns, such as square, rectangular, trapezoidal, triangular, diamond, parallelogram, circular, elliptical, pentagonal, hexagonal, and/or polygons with seven or more sides. The reinforcement elements comprising a reinforcement grid can all be of the same shape and size or can be of different shapes and sizes.

The diameter of a reinforcement element having a substantially circular cross-section can be from about 0.03 mm to about 0.50 mm in one embodiment, or from about 0.07 mm to about 0.30 mm in another embodiment, or from about 0.05 mm to about 1.0 mm in another embodiment, or from about 0.03 mm to about 1.0 mm in another embodiment. In another embodiment, the diameter of a reinforcement element having a substantially circular cross-section can be equivalent to a USP suture diameter from about size 8-0 to about size 0 in one embodiment, from about size 8-0 to about size 2 in another embodiment, from about size 8-0 to about size 2-0 in another embodiment.

The polymers that can be used to bond the reinforcement to the matrix include non-degradable polymers such as, but not limited to, polyesters; polyolefins, polycarbonates, polyurethanes, silicone, and any mixture thereof. The polymers that can be used to bond the reinforcement to the matrix include degradable polymers such as, but not limited to, copolymers of lactic acid, glycolic acid, lactide, glycolide, para-dioxanone, trimethylene carbonate, ε-caprolactone, polyoxaesters including polyoxaesters containing amido groups, polyanhydrides,and blends thereof. Aliphatic polyesters, for the purpose of this application, include polymers and copolymers of lactide (which includes lactic acid d-, l- and meso lactide), coplymers of ε-caprolactone and l-lactide, coplymers of ε-caprolactone and d/l-lactide, coplymers of ε-caprolactone and glycolide, ε-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, 6,6-dimethyl-1,4-dioxan-2-one, and any mixture thereof. The polymer can be in the form of pellets, sheets, or components that cross-link and are cured by heat or activated by catalyst. In one embodiment, the bonding polymer can be applied or pre-coated to the reinforcement or to the matrix by typical pre-pregging methods or by using solution processes such as dip coating or spraying. The bonding polymer can be subjected to compression molding or compressive molding by putting it in between the elastomeric matrix and the reinforcement and using compression molding and/or compressive molding conditions.

In another embodiment, annealing at elevated temperatures can also promote structural stabilization in cross-linked polyurethanes and the reinforcement fibers and enhance the long-term shelf-life stability of the device. In one embodiment, without being bound by any particular theory, annealing leads to relaxation of the stresses formed in the reticulated elastomeric matrix structure and/or in the reinforcement and/or in the adhesive.

The two ends of the polypropylene suture are then tied together to achieve a superior anterior pull of the hyoid and tongue base. The inferior wound is drained and both wounds are closed in layers. All patients were admitted for close observation overnight. Perioperative and postoperative care includes; steroids, intravenous antibiotics, anti-reflux medications, humidified oxygen, intravenous fluids, nasal trumpet, and pulse oximetry monitoring.

The most important part of surgical treatment of OSAHS is making a correct diagnosis of the site(s) of obstruction. The difficulty with OSAHS and tongue base obstruction has been the poor ability to grade severity and predict surgical success.

The advantages of the hyoid expansion and suspension are multi-fold. The splitting of the hyoid allows for potential lateral widening of the hypopharyngeal airway. Division of the infrahyoid musculature allows at least partial separation of the larynx from the hyoid and tongue base. This incomplete laryngeal drop can keep the inferior pulling force of intrathoracic structures from the hypopharyngeal structures separate. The negative pressures that occur during inspiration phase of respiration in an OSA patient can significantly contribute to further collapsing the flaccid structures of the upper airway.

Potential complications of hyoid suspension and expansion include airway obstruction, prolonged odynophagia, speech problems, neck hematoma, wound infection and intense cervical pain. Our experience however has unexpectedly shown very few problems. Pain and dysphagia/odynophagia are the most common complaints, but have all were resolved in reasonable time. Patients with prolonged dysphagia/odynophagia may benefit from instructions in a modified supraglottic swallow (tongue push and elevation with throat clearing) as mentioned by Woodson and Fujita. It is important to note that there were no airway or bleeding problems in the perioperative period. There were no suture or equipment problems as well.

Hyo-mandibular suspension and hyoid distraction-expansion procedures for tongue base obstruction has an important place in the surgical correction of tongue base and hypopharyngeal collapse in patients with severe OSA. This relatively minor external procedure is preferred over multiple stage transoral thermal ablation of the tongue base. It has been demonstrated that Hyoid expansion with interposition of a semirigid spacer implant is a novel surgical method that potentially enlarges the pharyngeal airspace in a lateral direction.

Preferably hyoid spacer 68 is cylindrical or substantially cylindrical in shape and has indentations that receive and hold ends of the hyoid segments. In another embodiment, the matrix can be cylindrical, substantially cylindrical or other shapes such as elliptical or helical but can substantially conform to the ends of the hyoid segments. In one embodiment, the matrix has a surface that has at least one at least partially spherical surface or at least partially curvilinear geometry that includes, for example, helical configurations, partially helical configurations, elliptical configurations, partially elliptical configurations, spherical configurations, partially spherical configurations, Optionally the matrix can be attached or affixed to the hyoid bone segments by one or more staples, sutures, clips, other mechanical means, and/or adhesive.

FIG. 7 is a schematic, cross-sectional representation of a hyoid spacer useful according to the invention. Typically, a hyoid spacer 70 will be a cylindrical or substantially cylindrical member or at least partially curvilinear of from about 30 to about 50 mm, preferably from about 35 to about 45 mm, in length. Each end 72 of spacer 70 has an annular recess 74 of from about 6 to about 14 mm, preferably from about 8 to about 12 mm, in depth. As shown in FIG. 7A, the annular recess 74 can have a wall thickness 76 of from about 1.5 to about 8 mm, preferably from about 3.5 to about 4.5 mm. In another embodiment, the annular recess 74 can have a wall thickness 76 of from about 3.0 to about 5.0 mm,

Different embodiments of tongue retractors are useful according to the invention. In an embodiment shown in FIGS. 8A and 8B, a tongue retractor 80 comprises a flat sheet 82 of biomedical grade polypropylane mesh between two flat sheets 84 of reticulated polymeric matrix. FIG. 8A is a lateral, somewhat cross-sectional view of tongue retractor 80. FIG. 8B is a top view of tongue retractor 80 where the shape and dimensions can be appreciated. Longitudinally extending side 88 has a length of from about 110 to about 130 mm, preferably from about 115 to about 125 mm, and width 90 is from about 5 to about 15 mm, preferably from about 8 to about 12 mm. The shorter lateral surface 92 is from about 10 to about 30 mm, preferably from about 15 to about 25 mm, in length, and each end surface 94 is from about 1 to about 3 mm, preferably from about 1.5 to about 2.5 mm, in length. Preferably the thickness of tongue retractor 80 is from about 1 to about 3 mm, preferably from about 1.5 to about 2.5 mm.

In another embodiment of a tongue retractor useful according to the invention shown in FIG. 9, a matrix protected suture configuration 98 comprises a reticulated polymeric matrix cylinder 100 with a suture 102 extending longitudinally therethrough. In another embodiment, reticulated polymeric matrix cylinder 100 is elastomeric and biodurable and resilient. Cylinder 100 has a length of from about 40 to about 80 mm, preferably from about 50 to about 70 mm, and its diameter is from about 2 to about 4 mm, preferably from about 2.5 to about 3.5 mm. The suture 102, such as, for example, 2-0 Ethibond suture, has a length of from about 100 to about 140 mm, preferably from about 110 to about 130 mm.

In a further embodiment of a tongue retractor useful according to the invention shown in FIGS. 10A and 10B, tongue retractor 110 comprises a single sheet 112 of reticulated polymeric matrix having sutures 114 extending therethrough. In another embodiment, reticulated polymeric single sheet 112 is elastomeric and biodurable and resilient. As shown in lateral view of FIG. 10A and the top view of FIG. 10B, sutures 114 extend through sheet 112 in parallel fashion in a longitudinal direction. Suture lines 116 and 118 represent lines of suture sewn into sheet 112 in longitudinal and perpendical directions, respectively. Suture lines 118 and suture 114 knot together at intersection points 120. Each suture 114 can be, for example, 2-0 Ethibond suture, whereas each of suture lines 116 and 118 can be, for example, 5-0 Ethibond suture.

Each end of each suture 114 extends from about 40 to about 80 mm, preferably from about 50 to about 70 mm, from sheet 112. Sheet 112 has a thickness of from about 1 to about 3 mm, preferably from about 1.5 to about 2.5 mm. The length of sheet 112 is from about 10 to about 30 mm, preferably from about 15 to about 25 mm, and the width of sheet 112 is from about 5 to about 15 mm, preferably from about 8 to about 12 mm. Each suture line 116 is from about 1 to about 3 mm, preferably from about 1.5 to about 2.5 mm, from the shorter edge 124 of sheet 112, and each suture line 118 is from about 2 to about 4 mm, preferably from about 2.5 to about 3.5 mm, from the longitudinal edge 126 of sheet 112.

### EXAMPLES

### Example 1

Patients were treated as described above with regard to FIGS. 1 to 6. The most common postoperative patient complaints included dysphagia, odynophagia and surgical wound pain. All patients were able to tolerate liquids and a soft diet at the time of discharge. No patients requested or required removal of the suspension sutures.

After 3 to 12 months of follow-up postoperatively, over 90% of the patients were subjectively improved by patient and/or spouse history. Symptomatic improvement included decrease in snoring, improved quality of sleep, and decreased daytime somnolence. Follow-up polysomnographic studies were performed 3 months to 1 year postoperatively in 52 patients. The patients that underwent hyo-mandibular suspension with or without hyoid expansion showed significant improvement in the mean RDI (Respiratory Disturbance Index) from 71.2 (±18.0) preoperatively to 28.4 (±16.8) postoperatively.

### Example 2

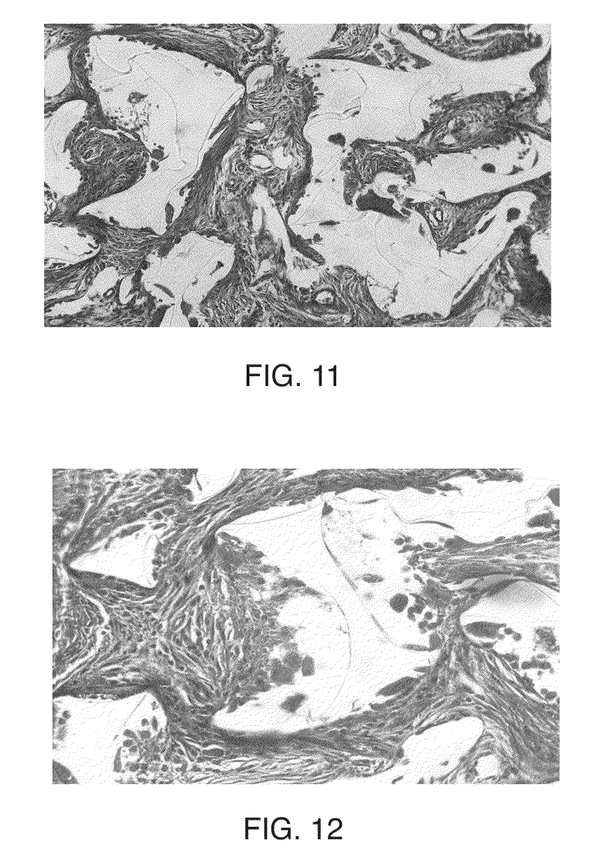

An incision was made in the anterior portion of the neck of a dog, and an implant comprised of reticulated elastomeric polycarbonate polyurethane-urea matrix was inserted into musculature at the base of the dog's tongue. A cavity was created, and the implant was secured with sutures in that position and left in place for three months. Fibrovascular tissue cells grew into the elastomeric matrix of the implant, as is shown in the micrograph slides shown in FIGS. 11 and 12. The implant comprises a netlike structure of unstained, short, narrow, birefringent synthetic fibers, and these foreign fibers are surrounded by individual histiocytes and (mmGCs) of foreign body type. These cells are between the device material and many small (˜0.2 mm to ˜0.4 mm) irregularly round masses of fibroblasts and collagen. Within these fibrous foci are arterioles, veins and capillaries of varied size and number. The fibrocytic reaction at the perimeter of the device extends into the interstitial tissues about 1.5 mm to 3 mm. This reactive process has encompassed some now atrophied skeletal muscle fibers.

Several skeletal muscle fibers in other, remote sites have contraction artifacts; this may have resulted from exposure to formalin very soon after the death of the animal when the muscle cells were still ‘irritable’ and capable of contracting. Only a very few plasma cells and no neutrophils (PMNs), unequivocal lymphocytes, or evidence of infection were found.

Morphologic Diagnosis: Chronic granulomatous foreign body reaction to synthetic material. The histologic features in these slides are consistent with the end (late) stage of the healing process. An additional change that might occur with more time would be contraction of the fibrous tissue as it matures.

Further reading in the area of the subject of the invention can be found as follows:

Chaban R, Cole P, Hoffstein V. Site of upper airway obstruction in patients with idiopathic obstructive sleep apnea. Laryngoscope 1988; 98: 641-647.

Chervin R D, Guilleminault C. Obstructive sleep apnea and related disorders. Neurol Clin 1996; 14(3): 583-609.

DeRowe, A, Gunther, E, Safaya, A, et al. Tongue base suspension for sleep disordered breathing: a new technique and device using a bone to soft tissue anchor. Anim Exp Prelim Clin Result.

Fujita S, Conway W, Zorick F, et al. Surgical correction of anatomic abnormalities in obstructive sleep apnea syndrome: uvulopalatopharyngoplasty. Otolaryngol Head Neck Surg 1981; 89: 923-934.

Fujita S . Pharyngeal surgery for obstructive sleep apnea and snoring. In: Snoring and Obstructive Sleep Apnea. Fairbanks DNF, et al., eds. Raven Press, New York; 1987, pp. 101-128.

Fujita S, Woodson B T, Clark J, et al. Laser midline glossectomy as a treatment for obstructive sleep apnea. Laryngoscope 1991; 101: 805-9.

Lowe A A, Fleetham J A, Adachi S, et al. Cephalometric and computed tomographic predictors of obstructive sleep apnea severity. Am J Orthod Dentofac Orthop 1995; 107: 589-595.

Lowe A A, Gionhaku N, Takeuchi K, et al. Three-dimensional CT reconstructions of tongue and airway in adult subjects with obstructive sleep apnea. Am J Orthod Dentofac Orthop 1986; 90: 364-374.

Rapoport D M, Garay S M, Goldring R M. Nasal CPAP in obstructive sleep apnea: mechanisms of action. Bull Eur Physiopath Resp 1983; 19: 616-620.

Riley R, Guilleminault C, Powell N, et al. Palatopharyngoplasty failure cephalometric roentgenograms and obstructive sleep apnea. Otolaryngol Head Neck Surg 1985; 93: 240-250.

Riley R W, Powell N B, Guilleminault C, et al. Maxillary mandibular and hyoid advancement: an alternative to tracheostomy in obstructive sleep apnea syndrome. Otolaryngol Head Neck Surg 1986; 94: 584-588.

Riley R W, Powell N B, Guilleminault C. Inferior sagittal osteotomy of the mandible with hyoid myotomy-suspension: a new procedure for obstructive sleep apnea. Otolaryngol Head Neck Surg 1986; 94: 589-593.

Riley R W, Powell N B, Guilleminault C. Maxillary mandibular and hyoid advancement for treatment of obstructive sleep apnea syndrome: a review of 40 patients. J Oral Maxillofac Surg 1990; 48: 20-26.

Riley R W, Powell N B, Guilleminault C. Maxillofacial surgery and obstructive sleep apnea: a review of 80 patients. Otolaryngol Head Neck Surg 1989; 101: 353-361.

Riley R W, Powell N B, Guilleminault C. Obstructive sleep apnea and the hyoid: a revised surgical procedure. Otolaryngol Head Neck Surg 1994; 111: 717-721.

Riley R W, Powell N B, Guilleminault C. Obstructive sleep apnea syndrome: a review of 306 consecutively treated surgical patients. Otolaryngol Head Neck Surg 1993; 108: 117-125.

Riley R W, Powell N B, Guilleminault C. Obstructive sleep apnea syndrome: a surgical protocol for dynamic upper airway reconstruction. J Oral Maxillofac Surg 1993; 51: 742-747.

Rojewski T E, Schuller D E, Clarke R W, et al. Video-endoscopic determination of the mechanisms of obstruction in obstructive sleep apnea. Otolaryngol Head Neck Surg 1984; 92: 127-131.

While illustrative embodiments of the invention have been described, it is, of course, understood that various modifications of the invention will be obvious to those of ordinary skill in the art. Such modifications are within the spirit and scope of the invention which is limited and defined only by the appended claims.

What is claiemd:

1. An improved method for surgically treating a sleep-related breathing disorder, comprising hyoid destruction/expansion and hyo-mandibular suspension, the improvement comprising an implant comprising a polypropylene and/or reticulated elastomeric biostable polyurethane matrix with the matrix being positioned between two or more segments of the destructed hyoid bone.

2. The method of claim 1 which also includes transoral or transcervical tongue suspension using similar implants.

3. The method of claim 1, wherein a single or multiple midline or paramedian incisions are made under a patient's chin;
soft tissues overlying the mandible are cleaned;
a first screw is inserted into the edge of the mandible;
a second screw is inserted into the inferior edge of the mandible;
a second horizontal incision is made over the body of the hyoid;
the infrahyoid muscles are separated from the body of the hyoid bone;
the hyoid is refracted and stabilized;
the sternohyoid and thyrohyoid muscles are detached from the body of the hyoid between the lesser cornuae;
the hyoid bone is divided midline or paramedian into two or more segments;
an absorbable or nonabsorbable, rigid or semirigid, implant is positioned between segments of the hyoid bone, whereby the hyoid bone is stabilized by the implant; and
sutures or portions of polypropylene and/or polyurethane matrix are passed around the hyoid bone segments and implant to connect to muscle still attached to the upper portion of the hyoid bone,
wherein the hypopharyngeal airway is expanded.

4. The method of claim 3, wherein the implant comprises a polypropylene and/or polyurethane matrix.

5. The method of claim 3, wherein the implant is cylindrical or substantially cylindrical with indentations at its respective ends.

6. The method of claim 3, wherein the implant substantially conforms to the substantially conform to the ends of the hyoid segments.

7. The method of claim 1, wherein the implant is attached to one or more hyoid bone segments with one or more staples, another mechanical means, or adhesive.

8. The method of claim 2, wherein the implant is attached to one or more hyoid bone segments with one or more staples, another mechanical means, or adhesive.
